(19) United States
(12) Patent Application Publication
(10) Pub. No.: US 2017/0333631 A1
(43) Pub. Date: Nov. 23, 2017

(54) DEVICE AND METHOD TO FACILITATE PLEURODESIS FOR MANAGEMENT OF FLUID DRAINAGE

(71) Applicant: NATIONAL JEWISH HEALTH, Denver, CO (US)

(72) Inventor: Ali I. MUSANI, Mequon, WI (US)

(21) Appl. No.: 15/521,923

(22) PCT Filed: Nov. 5, 2015
(86) PCT No.: PCT/US15/59305

§ 371 Date: Apr. 26, 2017

Related U.S. Application Data
(60) Provisional application No. 62/075,530, filed on Nov. 5, 2014.

(51) Int. Cl. A61M 5/19; A61M 5/20; A61M 5/31; A61M 5/315; A61M 5/178

(52) U.S. Cl. CPC A61M 5/19; A61M 5/3137; A61M 5/31596; A61M 5/20; A61M 2005/3125; A61M 2005/1787; A61M 2005/3128; A61M 2205/50; A61M 2210/101; A61M 2005/2026

(57) ABSTRACT

A device, method, and system are provided for treating pleural effusion by administration of a chemical agent through a pleural catheter to achieve pleurodesis. A first embodiment includes a dual barreled syringe to deliver the sclerosing agent; one barrel includes a sclerosing agent, and the other barrel includes a flushing fluid. A kit of the invention comprises a plurality of syringe devices each preloaded with a sclerosing agent matched with a presumptive diagnosis regarding the etiology of the pleural effusion to be treated. A device of the invention includes a handheld, disposable, electronic delivery device with preloaded chambers holding selected medicaments and flushing fluid. Automatic delivery of medicaments is achieved in which a microcontroller of the device controls evacuation of the selected chambers by one or more actuators that communicate with the chambers.

### FIELD OF THE INVENTION

The invention relates to methods of treating pleural effusion, and more particularly, to a device and method to facilitate pleurodesis.

### BACKRGOUND OF THE INVENTION

The pleural space lies between the visceral pleural layer that covers the surfaces of the lungs and a parietal pleural layer that covers the interior of the chest wall including the ribs and diaphragm. A thin film of fluid separates these two layers and provides a means to lubricate frictional contact between the surfaces of the lungs (visceral pleura) and the surrounding tissue (parietal pleura) as the lungs expand and contract during respiration. The collection of an excess amount of fluid between the pleural layers is referred to as pleural effusion. Pleural effusion can be associated with various conditions to include cancer, congestive heart failure, pneumonia, tuberculosis, pulmonary emboli, viral diseases, pancreatitis, and others.

The prompt removal of excess fluids from the pleural space is a first important step in the treatment of symptomatic pleural effusions. There are significant and serious health risks incurred by a patient who is not timely treated. There is an increased chance for morbidity and mortality when the pleural effusion taking place is associated with a severe disease. The fluid in the pleural space compresses the lung and may result in a partial or complete collapse of the lung. Lung collapse can lead to severe shortness of breath, cough and chest discomfort. Additionally, the excess collected fluid may become infected and therefore also cause further complications to the patient.

Pleurodesis is a common treatment for patients with recurrent symptomatic pleural effusions. Pleurodesis involves a chemical or mechanical irritation of the parietal and/or visceral layers of the pleura to collapse the gap or space between the visceral layers. More specifically,

pleurodesis involves the introduction of a sclerosing agent between the two pleural membranes, resulting in a scarring reaction to effectively join the layers to one another. The fusion or joining of the layers effectively eliminates the pleural space, thereby preventing fluid from collecting.

Known procedures for treatment include draining of the fluid by use of a chest catheter introduced into the pleural space, and then introducing a chemical irritant to cause the pleural layers to adhere to one another. Specific agents or compounds used for the chemical irritant may include talc, silver nitrate, tetracycline, doxycycline, and others. With the introduction of the irritant, scarring in the form of fibrous adhesions develop between the layers of the pleura. In order to achieve a pleurodesis for effectively preventing appreciable amounts of fluid from collecting in the pleural space, the sclerosing agent should be spread across a significant area of the pleural surfaces such that pockets do not develop between areas of adhered pleural layers. However, the scarring must not be so significant that it prevents the lungs from effectively expanding.

Particularly for patients who may have recurrent pleural effusions, removal of pleural fluid may be required numerous times. Although the removal of excessive pleural fluid may result in temporary relief and increased lung capacity, those patients with recurring pleural effusions in many cases require pleurodesis to significantly reduce the size of the pleural space.

Success rates vary for achieving adequate pleurodesis depending on the modality used. Currently, the most common method requires several days of hospitalization and placement of a chest tube to drain the fluid and to introduce chemical agents such as talc in the pleural space. For a significant number of patients, such as those with lung cancer, pleural effusion is associated with a very poor prognosis and the few remaining months of life for the patient may require numerous outpatient and/or inpatient procedures.

In order to reduce or eliminate multiple procedures to drain recurrent pleural effusions, or to prevent hospitalization to achieve pleurodesis, some commercial products have been developed to enable a caretaker to conduct drainage. Accordingly, treatment may be conducted at home under a physician's supervision. One example of a drainage catheter system includes the Pleurx® drainage catheter system. This particular system includes a soft, flexible silicone catheter that is placed in the pleural space by medical personnel. The flexible nature of the catheter enables it to conform to the pleural space, thereby minimizing patient discomfort. Large, smooth, fenestrations with beveled edges on the catheter promote drainage and help to avoid occlusions. An expandable cuff is located at the surgical entrance point in the patient's chest to hold the catheter in place. A safety valve is incorporated at the end of the catheter to prevent inadvertent passage of air or fluid back in either direction through the catheter. When drainage is required, a drainage tube is connected to the catheter by placing one end of the drainage tube carrying a stiff portion into the valve of the pleural catheter protruding from the patient's body. The other end of the drainage tube can be connected to a collecting receptacle such as a vacuum bottle. A vacuum bottle is pre-attached to the drainage tube in home drainage systems for the convenience of the patients.

Another example of a commercial drainage catheter system includes the Rocket® IPC pleural catheter. This system is similar to the Pleurx® drainage catheter system; however, certain components between the two systems differ, such as the particular safety valves and clamps used.

Indwelling pleural catheters can be placed in an outpatient procedure so a patient can be discharged the same day thus precluding hospitalization. These patients are then managed entirely on an outpatient basis including removal when spontaneous pleurodesis is achieved or when the catheter is no longer necessary for drainage purposes. According to research over the last 10 years or so, approximately 50-60% of patients with malignant pleural effusions achieve spontaneous pleurodesis (referred to as auto pleurodesis) in 40-50 days without any intervention except having the pleural catheter in the pleural space drained periodically, such as every other day. While pleural catheters can provide an outpatient management of pleural effusions, the length of time the catheters are required to remain in the patients is not desirable. Additionally, most patients prefer out patient management as opposed to a several day hospitalization with a chest tube or a surgical procedure.

Therefore, there is a need to administer sclerosing agents to achieve pleurodesis without requiring the patient to be admitted to a hospital. There is also a need to provide administration of the sclerosing agents in a safe, simple, and predictable procedure that does not require the services of a registered nurse or medical doctor. There is yet further a need to provide administration of sclerosing agents tailored for each patient according to the particular etiology associated with the pleural effusions taking place.

### SUMMARY OF THE INVENTION

According to one embodiment of the invention, it includes a method of treating pleural effusions. According to the method, one or more treatments are conducted for removing fluid from the pleural space of a patient. Fluid is removed by placing a pleural catheter within the pleural space of the patient. The method further includes achieving pleurodesis by administration of a chemical irritant such as a sclerosing agent. Preferably, administration is achieved by use of a dual chambered or dual barreled syringe in which one barrel holds a sclerosing agent, and the other barrel holds a flushing fluid such as saline solution.

In another embodiment of the invention, it includes a dual barreled syringe device that can be used by a caretaker to deliver a sclerosing agent into the pleural catheter. The device includes two separate syringes joined to one another along their lengths, and each syringe having a plunger to selectively introduce the sclerosing agent and flushing fluid. According to one configuration of this dual barreled syringe embodiment, the device includes an integral connecting manifold providing corresponding passageways or legs for receiving the sclerosing agent and flushing fluid, and a downstream conduit that allows passage of the sclerosing agent followed by flushing fluid into the pleural catheter. According to another configuration of this embodiment, the connecting manifold is a separate element from the dual barreled syringe in which the connecting manifold is attached to the distal discharge ends of the syringe barrels by a friction type connection. Because two separate syringes are used with separately operable plungers, selected amounts of the sclerosing agent and flushing fluid can be delivered, as well as the selected sequence in which the sclerosing agent and flushing fluid are delivered.

In another embodiment of the invention, it includes a kit comprising a plurality of dual barreled syringe devices in which the types and amounts of sclerosing agents are matched to a presumptive diagnosis regarding the etiology of the pleural effusion to be addressed by the pleurodesis. In this regard, the devices contain the appropriate amounts of sclerosing agents and flushing fluid to be administered to a patient in a predetermined regimen corresponding to the nature of the pleural effusion being addressed. For example, the kit may include multiple devices, each marked with a symbol (such as letters, numbers, symbols, or combinations thereof) that correspond to the date/time in which the sclerosing agent is to be administered to the patient, or other instructional information regarding use of the kit. The kit further includes general instructions as to the particular syringe devices that should be used for administering the sclerosing agent, the order in which multiple syringe devices should be used (if multiple syringe devices are required for a particular regimen), the generally designated dates and/or times in which administration should take place, and the selected amounts in each syringe device that should be used according to the prescribed treatment regimen.

According to another preferred embodiment, a portable handheld electronic delivery device is provided that is capable of delivering selected quantities of medicaments and fluids including sclerosing agents, analgesics and flushing fluids in an automatic, predetermined sequence to a pleural catheter or other types of selected catheters. The device has a housing which contains the device components. The housing may be sealed to prevent access to the medicaments and fluids that are pre-loaded within corresponding delivery chambers. The contents within the plurality of chambers may be selected according to a desired treatment protocol and therefore, the device is not limited to any particular combination of medicaments or fluids. During assembly of the device, delivery chambers are preloaded with selected medicaments and/or fluids according to a desired treatment protocol. An actuator is used for each chamber to evacuate the contents therein, or a single actuator can be used to selectively actuate the chambers sequentially. One end of each of the chambers communicates with corresponding delivery passageways so that fluid may be transferred to the connected catheter system. A check valve may be used for each of the chambers to prevent backflow of fluid, and a delivery valve may be incorporated near the fluid discharge point of the device to control fluid leaving the device. A start or delivery button can be used as the means to initiate fluid delivery. A microcontroller within the device sends control signals to the actuator(s) for the desired sequence of delivery of fluid. The microcontroller can be preprogrammed for a wide range of delivery sequences for selected ones of the plurality of medicament/fluid containing chambers. With the electronic delivery device, delivery of fluid to the catheter system is automatic, and the contents within the device are protected from tampering and damage.

Considering each patient may have special requirements regarding the type, frequency, and amounts of sclerosing agent that should be administered, the invention further includes a method in which the kit has a predetermined number of syringe devices with one or more types of sclerosing agents that can be used for administration to the vast majority of all patients that suffer from pleural effusions. The method further includes determining an appropriate pleurodesis treatment protocol in which unique instructions for the treatment are generated for each patient based upon the special circumstances surrounding the pleural effusion to be treated. The instructions may include descriptions of the particular syringe device(s) to be used in the treatment, the time(s) in which treatments should occur, the amounts of the sclerosing agent(s) to be delivered in each treatment, and follow-up instructions to confirm pleurodesis has been successfully achieved. The instructions may be automatically generated from a computer readable medium, such as software program, with the appropriate logic incorporated therein that generates the desired treatment instructions to be conducted based upon the particular patient, and circumstances surrounding the pleural effusion to be treated. Further, the instructions may contain specific references to the symbols associated with each of the devices so that a caregiver has clear, easy to follow instructions regarding which syringe device(s) to use, at the prescribed time(s), and the specific amount(s) to be administered.

According to one preferred method of the invention, administration of a sclerosing agent is achieved by several small daily doses as opposed to a single larger dose. It is believed that administering a series of smaller daily doses provides a more comfortable solution for the patient, yet still achieves effective pleurodesis. In one particular example of this method, five daily infusions can be administered via a pleural catheter communicating with the pleural space. Further, a kit of the invention may include five syringe devices in which each syringe is preloaded with a prescribed daily infusion. In this way, the kit provides a complete solution for achieving the daily doses.

In accordance with the above described features of the invention, in one aspect, the invention may be considered a syringe device comprising (i) a first syringe barrel having a chamber defining a first volume therein; (ii) a first plunger inserted in the barrel; (iii) a second syringe barrel having a chamber defining a second volume therein; (iv) a second plunger inserted in the second barrel; (v) a manifold attached at a distal end of the device, the manifold having a first leg communicating with the chamber of the first syringe barrel, a second leg communicating with the chamber of the second syringe barrel, and a common conduit interconnecting the first and second legs, wherein the first syringe barrel has first contents therein, the second syringe barrel has second contents therein, the syringe device being operated to first evacuate the first contents from the first syringe barrel by depressing the plunger, and then operated to next evacuate the second contents from the second syringe barrel.

According to this first aspect of the invention, the first syringe barrel can be marked to identify the contents therein including a designation regarding an order in which the first syringe barrel is to be evacuated for administration to a patient; and the second syringe barrel can also be marked to identify the contents therein including a designation regarding an order in which the second syringe barrel is to be evacuated for administration to the patient. Further according to this first aspect of the invention, it may further include a lock element positioned between a thumb rest of the first syringe barrel and a proximal end of the first syringe barrel.

In another aspect of the invention, it may include a method of treating pleural effusion of a patient, comprising: (i) placing a pleural catheter in the pleural space of the patient; (ii) draining an amount of pleural fluid from the patient through the catheter; (iii) connecting a dual barreled syringe device in line with a drainage portion of the catheter, the dual barreled syringe having; (a) a first syringe barrel having a chamber defining a first volume therein; (b) a first plunger inserted in the barrel; (c) a second syringe barrel having a chamber defining a second volume therein; (d) a second plunger inserted in the second barrel; (e) a manifold attached at a distal end of the device, the manifold having a first leg communicating with the chamber of the first syringe barrel, a second leg communicating with the chamber of the second syringe barrel, and a common conduit interconnecting the first and second legs; (iv) administering an amount of sclerosing agent by first depressing the first plunger having the sclerosing agent therein causing the sclerosing agent to travel through one of the legs of the manifold, into the common conduit, and into the catheter; (v) next flushing an amount of flushing fluid through the manifold and catheter by depressing the second plunger having the flushing fluid therein causing the flushing fluid to travel through the other leg of the manifold into the catheter; and (vi) delivering the sclerosing agent through the catheter into the pleural space of the patient in which the flushing fluid follows the sclerosing agent to therefore flush the catheter of the sclerosing agent.

This second aspect of the invention may include administering in a predetermined sequence in which the plungers of the first and second barrels are selectively activated according to instructions in which the first and second barrels include markings corresponding to the predetermined sequence. This second aspect may further include selectively activating in the predetermined sequence further includes selectively activating the plungers to deliver predetermined amounts of the sclerosing agent from the first barrel and predetermined amounts of the flushing fluid from the second barrel.

According to another aspect of the invention, it may be considered a kit especially adapted for use in administering sclerosing agents for treatment of pleural effusions, the kit comprising: (i) a plurality of syringe devices, each syringe device comprising: (a) a first syringe barrel having a chamber defining a first volume therein; (b) a first plunger inserted in the barrel; (c) a second syringe barrel having a chamber defining a second volume therein; (d) a second plunger inserted in the second barrel; (e) a manifold attached at a distal end of the device, the manifold having a first leg communicating with the chamber of the first syringe barrel, a second leg communicating with the chamber of the second syringe barrel, and a common conduit interconnecting the first and second legs allowing for passage of components passing through the legs from the respective chambers of the first and second syringe barrels; (ii) a sclerosing agent loaded in the first syringe barrel for use in pleurodesis, a flushing fluid loaded in the second syringe barrel for use in flushing a catheter through which the sclerosing agent is delivered to a patient; and (iii) instructions provided for each kit after the patient has been diagnosed with a pleural effusion to be treated, the instructions including directions for a caregiver to administer a selected amount of at least one sclerosing agent over a selected time period.

The kit may further include a description for administering the sclerosing agent in a predetermined sequence in which the plungers of the first and second barrels are selectively activated according to the instructions in which the first and second barrels include markings corresponding to the predetermined sequence. The instructions may further include a description for administering according to the predetermined sequence in which the plungers are activated to deliver predetermined amounts of the sclerosing agent and flushing fluid from the first and second barrels.

In yet another aspect of the invention, it includes a method for facilitating remote treatment of pleural effusion of a patient, the method comprising: (i) providing at least two computing devices and computer coded instructions in the form of computer readable mediums associated with said at least two computing devices for at least determining dosages with respect to a sclerosing agent to be administered to a patient for treatment; (ii) a first computing device of said at least two computing devices located at a medical treatment facility, including at least one of a hospital, clinic, or caregiver office, and a second computing device of said at least two computing devices located remote from the medical treatment facility at a patient location; (iii) providing a kit to the patient, the kit comprising: a plurality of syringe devices, each syringe device comprising: (a) a first syringe barrel having a chamber defining a first volume therein; (b) a first plunger inserted in the barrel; (c) a second syringe barrel having a chamber defining a second volume therein; (d) a second plunger inserted in the second barrel; (e) a manifold attached at a distal end of the device, the manifold having a first leg communicating with the chamber of the first syringe barrel, a second leg communicating with the chamber of the second syringe barrel, and a common conduit interconnecting the first and second legs allowing for passage of components passing through the legs from the respective chambers of the first and second syringe barrels; (iv) loading a sclerosing agent in the first syringe barrel for use in achieving pleurodesis, a flushing fluid loaded in the second syringe barrel for use in flushing a catheter through which the sclerosing agent is delivered to a patient; (v) instructions provided for each kit including directions for use of the kit to administer at least one sclerosing agent; (vi) obtaining information from the patient including a present health status of the patient by said second computing device communicating with said first computing device; (vii) determining a preferred pleural effusion treatment protocol from said computer coded instructions based upon consideration of the present health status of the patient, the computer coded instructions including a plurality of user menu options to select information corresponding to the health status of the patient and capabilities of the kit provided to the patient for determining the treatment protocol; (viii) generating a set of instructions for the protocol by said computer coded instructions including a selected amount and type of sclerosing agent to be administered to the patient to achieve pleurodesis; and (ix) communicating the set of instructions to a caregiver or to the patient.

This method for facilitating remote treatment of pleural effusion of a patient may further include (i) wherein the first computing device includes at least one of a personal computer, a smartphone, or a tablet computer, (ii) wherein the second computing device includes at least one of a personal computer, a smartphone, or a tablet computer, (iii) wherein the present health status of the patient includes vital signs, (iv) wherein the computer coded instructions include a computer readable medium such as software program residing in respective memory elements of the first and second computing devices, the software program being executed by respective processors of the first and second computing devices, (v) wherein a determined preferred pleural effusion treatment protocol from said determining step further includes instructions for a selected type or selected types of sclerosing agents to be administered from the kit, (vi) wherein a determined preferred pleural effusion treatment protocol from said determining step further includes instructions for different times in which a sclerosing agent is to be administered over a period of time, or (vii) wherein a subsequent health status of the patient is communicated from the second computing device to the first computing device after the patient is administered with the sclerosing agent.

According to another aspect of the invention, it may include An electronic delivery device for delivering selected sclerosing agents for treatment of pleural effusions, comprising: (i) a housing; (ii) a plurality of chambers placed within the housing, each of the chambers having a selected medicament, analgesic and/or flushing fluid preloaded therein; (iii) at least one actuator communicating with at least one of the chambers for evacuating contents of a selected chamber; (iv) at least one fluid passageway connected to the plurality of chambers for transferring contents of the chambers out from the device; and (v) a microcontroller communicating with the at least one actuator for evacuating contents within a selected chamber by action of the actuator.

Other aspect of the device may further include: a start button to initiate a sequence of fluid delivery as controlled by the microcontroller; a check valve associated with at least one of the chambers to prevent backflow; a fluid delivery valve communicating with the plurality of chambers to control flow of fluid from the device; and wherein the microcontroller is programmed for operation to enable delivery of a selected sequence of medicaments and flushing fluid from the chambers.

In yet another aspect of the invention, it may include a method of treating pleural effusion of a patient, comprising: (i) placing a pleural catheter in the pleural space of the patient; (ii) draining an amount of pleural fluid from the patient through the catheter; (iii) connecting a dual barreled syringe device in line with a drainage portion of the catheter, the dual barreled syringe having; a first syringe barrel having a chamber defining a first volume therein, a first plunger inserted in the barrel, a second syringe barrel having a chamber defining a second volume therein, a second plunger inserted in the second barrel, a manifold attached at a distal end of the device, the manifold having a first leg communicating with the chamber of the first syringe barrel, a second leg communicating with the chamber of the second syringe barrel, and a common conduit interconnecting the first and second legs; (iv) administering a first daily amount of sclerosing agent by depressing the first plunger having the sclerosing agent therein causing the sclerosing agent to travel through one of the legs of the manifold, into the common conduit, and into the catheter and flushing an amount of flushing fluid through the manifold and catheter by depressing the second plunger having the flushing fluid therein causing the flushing fluid to travel through the other leg of the manifold into the catheter; (v) delivering the sclerosing agent through the catheter into the pleural space of the patient so the flushing fluid follows the sclerosing agent to flush the catheter of the sclerosing agent; and (vi) repeating steps (iv) and (v) daily to deliver subsequent amounts of the sclerosing agent. Further according to this method, it may include executing five daily deliveries in which each delivery of the sclerosing agent may be in selected quantities/volumes in a solution containing the sclerosing agent.

Other features and advantages of the invention will become apparent from review of the following drawings, taken in conjunction with the detailed description.

### BRIEF DESCRIPTION OF THE DRAWINGS

FIG. 1 illustrates a perspective view of a dual barreled syringe device according to an embodiment of the invention;

FIG. 2 illustrates a perspective view of another dual barreled syringe device according to another embodiment of the invention;

FIG. 3 illustrates a schematic diagram of a pleural catheter system and a dual barreled syringe device according to another aspect of the invention; and

FIG. 4 illustrates a schematic diagram of another embodiment of the invention in the form of a portable handheld electronic delivery device which is capable of delivering selected quantities of sclerosing agents and flushing fluids in a predetermined sequence to a pleural catheter or other types of selected catheters.

### DETAILED DESCRIPTION

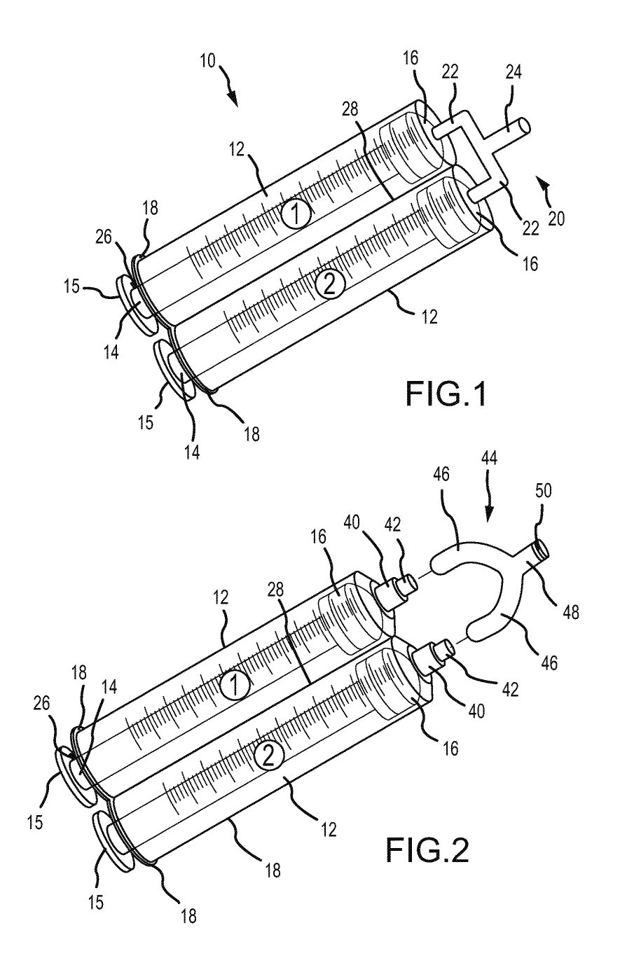

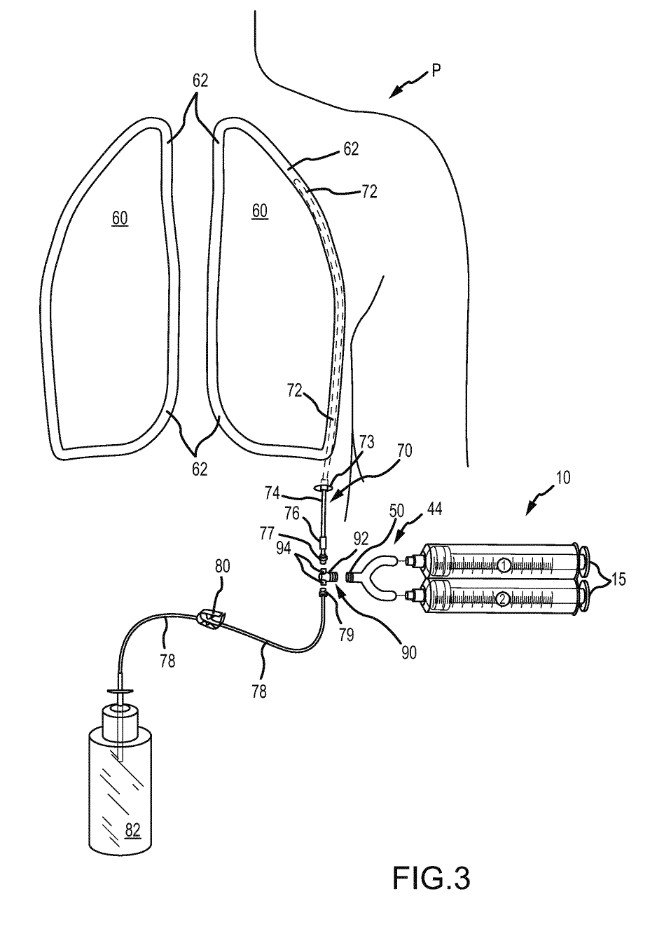

Referring to FIG. 1 in a first embodiment of the invention, it includes a dual barreled syringe device 10 to deliver a sclerosing agent into a drainage device, such as a pleural catheter. As shown in FIG. 3 and discussed below, the sclerosing agent is forcibly delivered through the catheter into the pleural space of a patient. The patient may be positioned so that delivery of the sclerosing agent is successfully achieved into the pleural space to contact the maximum surface areas of the pleural tissues. As shown in FIG. 1, the device 10 includes two separate syringes joined to one another along their length, each syringe having their own plungers to introduce the sclerosing agent and flushing fluid. More specifically, the device 10 includes two side-by-side and joined barrels 12, and each barrel 12 having a corresponding plunger 14. Other details illustrated in FIG. 1 include respective thumb rests 15 for each plunger 14, and respective plunger heads 16 that are sealed with respect to the interior surface of the barrels 12 for selective evacuation of contents within the chambers of the respective barrels. A proximal flange 18 is provided for both of the barrels 12 in order to provide a gripping or holding surface when operating the plungers 14. A plunger lock 26 can be used to lock the position for selected or each plunger 14 as shown. Each of the barrels has corresponding graduations or markings that show the amount of material in each barrel. The barrels are illustrated as having common cylindrical shapes and joined along facing surfaces of the barrels. However, it shall be understood that the shape of the barrels, as well as the interface or connection between the barrels can be modified in shape to enhance ergonomics for the user. Further, although the barrels are illustrated as having substantially equal sizes and shapes, it is also contemplated that the barrels may have different sizes and shapes to include differing volumes for retaining selected materials in the chambers.

According to one configuration of this first embodiment, the device includes an integral connecting manifold 20 providing corresponding passageways or legs for receiving materials within the barrels, such as the sclerosing agent and the flushing fluid, and a conduit for delivering the sclerosing agent and flushing fluid into the pleural catheter. More specifically, the integral connecting manifold 20 is illustrated with two passageways or legs 22, one end of one of the legs communicating with the interior volume or chamber of one the barrels, and one end of the other leg communicating with the interior volume or chamber of the other barrel. The opposite ends of the legs are directed towards one another and are joined at a common conduit 24.

Referring to FIG. 2, according to another embodiment, the connecting manifold 44 is shown as a separate element from the dual barreled syringe. More specifically, FIG. 2 illustrates the dual syringe 10 in which the distal ends of the barrels 12 each include a reduced diameter housing 40. Each barrel 12 further includes respective tip extensions 42 that frictionally engage with corresponding legs 46 of the manifold 44. The legs 46 are directed toward one another at their opposite ends, and are joined at the common conduit 48. The manifold 44 is also illustrated with a threaded connection 50 at the free end of the conduit 48.

FIGS. 1 and 2 also show the barrels 12 being marked with numbers (1 and 2), indicating for example, the contents loaded in the barrels, the sequence in which the contents should be administered, and/or other identifying information.

Referring to FIG. 3, a schematic diagram shows a pleural catheter system and a dual barreled syringe device according to another aspect of the invention. In particular, FIG. 3 shows a patient P with lungs 60 and pleural spaces 62 between the lungs and the surrounding tissues. A distal portion 72 of a pleural catheter 70 is inserted through an incision 73, and the distal portion 72 resides within the pleural space 62. A proximal portion 74 of the pleural catheter 70 extends through the incision 73 and is exposed. A cuff or other resilient expanding device (not shown) may be placed over the proximal portion 74 to effectively seal and close the incision 73 and to seal the catheter extending through the cuff. The free end of the proximal portion 74 may attach to a safety valve 76 to prevent air or fluid from inadvertently traveling back through the catheter. During a procedure to drain fluid, a fitting 77 located downstream of the safety valve 76 may be directly connected to a corresponding fitting 79 of a drainage tube 78. The opposite end of the drainage tube 78 connects to a vacuum bottle 82 to receive drained fluid from the patient. A clamp 80 may be attached to the drainage tube 78 in order to control flow of fluid through the drainage tube 70.

FIG. 3 also illustrates how the dual barreled syringe may be integrated in the catheter system. Specifically, FIG. 3 shows the dual barreled syringe of FIG. 2 having the separable manifold 44. A T-connector 90 can be used to place the dual barreled syringe 10 in fluid communication with the catheter system. As shown, the T-connector has two opposing legs or ends 94 that interconnect the fitting 79 of the drainage tube 78 with the fitting 77 of the catheter 70. The leg 92 is attached to the threaded end 50 of the manifold 44. In this configuration, a caregiver may commence administration of the sclerosing agent by depressing the thumb rests 15 of the syringes to deliver the prescribed amounts of materials. The construction of the device 10 allows for delivery of two compositions or fluids, and this is particularly advantageous for sclerosing agents that should be followed with a flushing fluid. In the same way, the dual barreled syringe of FIG. 1 with the manifold 20 may be used for administering the sclerosing agent by connecting the conduit 24 to the end 92 of the manifold 90.

Although FIG. 3 has been described with respect to a threaded connection between the leg 92 of the T-connector and the threaded end 50 of the manifold, it is also contemplated that other types of connections can be used, such as a friction fit connection. Further, the ends of the legs 94 for the T-connector may also incorporate a matching type of structure for connection to the catheter system. Some catheter systems use nonstandard fittings or connections, and therefore, it is specifically contemplated within the present invention that the T-connector be adapted for connection to the particular catheter system utilized employed. After administering the sclerosing agent, the manifold 20/44 is disconnected from the end 92 of the manifold 90, and the end 92 may be capped.

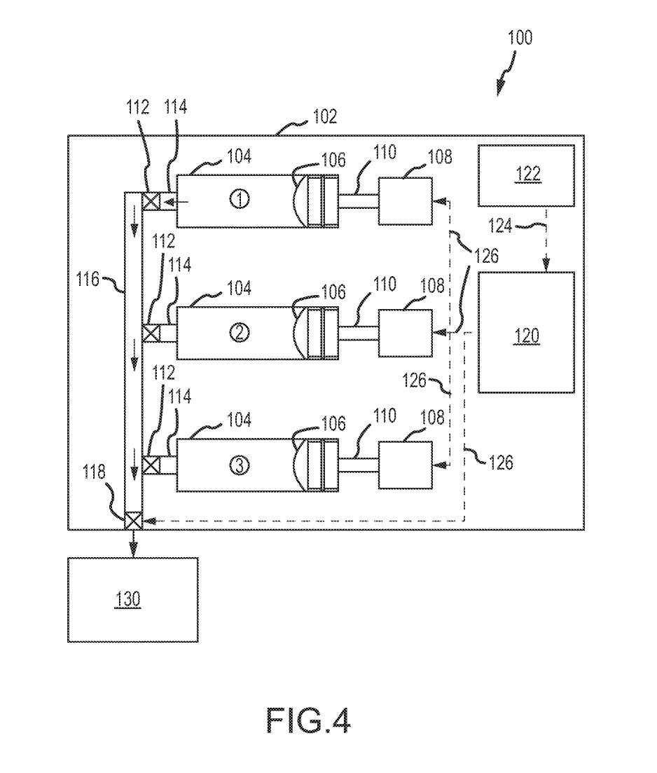

Referring to FIG. 4, another preferred embodiment of the invention is illustrated in the form of a portable handheld electronic delivery device 100 that is capable of delivering selected quantities of sclerosing agents, analgesic and/or flushing fluids in a predetermined sequence to a pleural catheter or other types of selected catheters. The device 100 has a housing 102 which contains the device components. The housing may be sealed to prevent access to the sclerosing agents, and/or analgesic or flushing fluids that are pre-loaded within the corresponding delivery chambers 104. In the example of FIG. 4, three chambers 104 are illustrated, each one containing a selected sclerosing agent, flushing fluid, or other medicament. One end of each of the chambers 104 communicates with a delivery passageway 114, which in turn communicates with a check valve 112, and then communicates with a delivery manifold 116. A flow control valve 118 may be used to control flow of fluid from the device to a selected catheter system 130, such as a pleural catheter.

Means are provided to evacuate contents within the chambers, and FIG. 4 illustrates an actuator 108 which may linearly move a plunger rod 110. The distal end of the plunger rod 110 attached to a plunger head 106, the rod and head which may be of similar construction to a plunger and plunger head of a syringe. A microcontroller 120 sends control signals to selected components, to include the actuators 108 and flow control valve 118. Operation of the device is controlled initially by a start or delivery button 122. Once triggered/depressed, a control signal 124 is sent to the microcontroller 120 to execute a predetermined and sequenced delivery of contents within the chambers 104 through the delivery valve 118. The three chambers 104 in FIG. 4 are marked sequentially as 1, 2, and 3, indicating that the sequenced delivery for this device results in three separate and sequential deliveries of contents within the chambers. More specifically, a first control signal activates the actuator 108 such that the contents within the chamber marked 1 is evacuated, a next control signal activates the next actuator 108 to evacuate contents within the chamber marked 2, and a final control signal activates the final actuator 108 to evacuate contents within the chamber marked 3. While the preferred embodiment of FIG. 4 illustrates three separate actuators 108, is also contemplated that there could be a single actuator that could control multiple chambers. For example, the actuator could have multiple actuating elements which could separately contact or otherwise control the chambers for the desired delivery sequence.

The first and/or second chambers may have a solution containing a sclerosing agent and/or analgesic, while the third chamber may have a flushing fluid. It should be understood that the microcontroller can be pre-programmed for execution of sequential or simultaneous delivery of one or more selected contents within selected chambers. Further, the microcontroller also provides variable and incremental control for each of the actuators such that the content of each chamber may be fully evacuated, or alternatively, it may be desired to only deliver selected amounts/volumes of medicaments within one or more of the chambers. In this regard, the microcontroller can control and execute a nearly limitless combination of delivery amounts/volumes and sequences among a plurality of chambers. The device 100 may be disposable. Therefore, a caregiver or patient has no further work associated with the device once the administration of the fluids/medicaments is complete.

One particular advantage of the preloaded, disposable device 100 is that it greatly simplifies administration of the sclerosing agent in an automatic fashion so that a caregiver or patient does not have to manually manipulate syringe devices. Further, the medicaments within the device are not accessible, and therefore, cannot be tampered with which may otherwise jeopardize the desired treatment protocol. Further, preloading of the chambers with desired medicaments also eliminates the possibility of error with respect to quality/volumes of medicaments, as well eliminating potential errors associated with the failure to fully evacuate the contents of the chamber; as compared to a syringe device in which there is a greater possibility of error in not fully evacuating a syringe body. Further, use of a single button is all that is required to set into motion the delivery of fluid without further commands or other manual manipulations or interventions.

According to one preferred method of the invention, the sclerosing agent is administered in several small daily doses as opposed to a single larger dose. Administering a series of smaller daily doses may provide a more comfortable solution for the patient, yet still achieves effective pleurodesis. In one particular example of this method, five daily infusions can be administered via a pleural catheter communicating with the pleural space. Further, a kit of the invention may include at least five syringe devices in which each syringe is preloaded with a prescribed daily infusion. In this way, the kit provides a complete solution for achieving the daily doses.

According to another embodiment of the invention, it includes a kit comprising a plurality of dual barreled syringe devices in which the types and amounts of sclerosing agents and flushing fluids are matched to a presumptive diagnosis regarding the etiology of the pleural effusion to be addressed by the pleurodesis. The syringe devices are preconfigured to hold the appropriate amounts of sclerosing agents and flushing fluids to be administered to a patient in a predetermined regimen corresponding to the nature of the pleural effusion being addressed. The kit may include multiple syringe devices, each marked with a symbol (such as a letter or number) that corresponds to the date/time in which the sclerosing agent is to be administered to the patient. The kit further may further include general instructions as to the particular syringe devices that should be used for administering the sclerosing agent, the order in which multiple syringe devices should be used, the generally designated dates and/or times in which administration should take place, and the selected amounts in each syringe device that should be used according to the prescribed treatment regimen. According to a particular treatment regimen, a caregiver may be required to administer the sclerosing agent from one or more syringe devices. The instructions will dictate which syringe device to use, the quantity of sclerosing agent to be evacuated into the catheter, the time in which this should occur, and the sequence in which agent and flushing fluid should be administered, assuming more than one type of sclerosing agent is required. According to some regimens, this may require use of multiple syringe devices over a period of days, depending upon how the patient responds to the treatment. The treatment regimen can be modified based upon a continued evaluation the patient after a first administration of the sclerosing agent has been conducted.

The kit may be especially configured to hold a predetermined number of syringe devices with one or more types of sclerosing agents that can be used for effectively treating most patients that suffer from pleural effusions. The method in this regard further includes determining an appropriate pleurodesis treatment protocol in which unique instructions for the treatment are generated for each patient based upon the special circumstances surrounding the pleural effusion to be treated. The instructions may include a description of the particular syringe device(s) in the kit to be used in the treatment, the time(s) in which treatments should occur, the amounts of the sclerosing agent(s) to be delivered in each treatment, and follow-up instructions to confirm pleurodesis has been successfully achieved. Markings may be placed on the syringe devices corresponding to the prescribed treatment regimen. For example in FIGS. 1 and 2, the numerical markings on the barrels 12 (the numbers 1 and 2), may correspond to the order in which the contents should be administered or other corresponding information as prescribed.

The instructions may be automatically generated from a computer readable medium such as a software program with the appropriate logic incorporated therein to generate the desired treatment to be conducted based upon the type of patient, and circumstances surrounding the pleural effusion to be treated. Further, the instructions may contain specific references to the symbols associated with each of the devices so that a caregiver has clear, easy to follow instructions regarding which syringe device(s) to use, at the prescribed time(s), and the specific amount(s) to be administered. It is further contemplated that the method may include remote feedback from the caregiver or patient that is sent to the hospital or clinic that has authorized the procedure, in which data is sent back to the authorizing organization regarding the status of the patient after receiving one or more administrations of the sclerosing agent. For example, the patient's vital signs, lung capacity, and other statistics may be sent electronically back to the authorizing organization, and then a modified protocol may be prescribed according to the specific type of response that the patient exhibits. The use of a single preconfigured kit can be used, along with some degree of automation with respect to generation of unique instructions for each caregiver, to accomplish treatment for patients suffering from diverse types of pleural effusions.

According to another preferred embodiment of the invention, a method is provided to for facilitating remote treatment of pleural effusion of a patient. In this method, communications are made between a medical treatment facility and a remote location where the patient is located. The patient is provided the kit including instructions in the kit for basic use of the kit to administer a sclerosing agent and flushing the catheter to deliver the sclerosing agent. A health status of the patient is confirmed prior to determining the type and amount of sclerosing agent to be administered. In this regard, the patient may be remotely monitored for vital signs, and these vital signs are communicated from the patient to the medical treatment facility. The method of communication may be by the patient's smartphone, home computer, or tablet computer. This communication may therefore be achieved by a wireless telephone transmission, or by an electronic transmission over the Internet. A computer program determines a treatment protocol based upon previous information obtained regarding the patient, and the current health status of the patient. The preferred treatment protocol is then communicated to the patient by a computing device located at the medical treatment facility. The treatment protocol includes instructions for use of the kit to deliver the type and amount of sclerosing agent in the kit. The kit may include one or more types and amounts of sclerosing agents in pre-designated syringe devices to account for different types of procedures that may be required for different patients. The instructions may further include times and dates in which additional deliveries of sclerosing agents should be administered over time. The health status of the patient can be continually monitored remotely to determine if additional sclerosing agents need to be delivered over time.

One clear advantage of this method is to eliminate the need for the patient to travel to a medical treatment facility to receive treatment, which also avoids the cost and time of having to admit, host, and discharge the patient. Particularly for those patients who may have terminal illnesses, the ability to provide in-home treatment is advantageous.

Although the invention has been described with respect to one or more preferred embodiments, it shall be understood that other changes and modifications may be made to the invention commensurate with the scope of the claims appended hereto.

What is claimed is:

1. A syringe device comprising:
a first syringe barrel having a chamber defining a first volume therein;
a first plunger inserted in the barrel;
a second syringe barrel having a chamber defining a second volume therein;
a second plunger inserted in the second barrel;
a manifold attached at a distal end of the device, the manifold having a first leg communicating with the chamber of the first syringe barrel, a second leg communicating with the chamber of the second syringe barrel, and a common conduit interconnecting the first and second legs, wherein said first syringe barrel has first contents therein, said second syringe barrel has second contents therein, said syringe device being operable to first evacuate the first contents from said first syringe barrel by depressing said plunger, and then operable to next evacuate the second contents from the second syringe barrel.

2. A syringe device, as claimed in claim 1, wherein:
said first syringe barrel is marked identifying the contents therein, and including a designation regarding an order in which the first syringe barrel is to be evacuated for administration to a patient; and
said second syringe barrel is marked identifying the contents therein, and including a designation regarding an order in which the second syringe barrel is to be evacuated for administration to the patient.

3. A syringe device, as claimed in claim 1, further including:
a lock element positioned between a thumb rest of said first syringe barrel and a proximal end of said first syringe barrel.

4-6. (canceled)

7. A kit especially adapted for use in administering sclerosing agents for treatment of pleural effusions, the kit comprising:
a plurality of syringe devices, each syringe device comprising: (i) a first syringe barrel having a chamber defining a first volume therein; (ii) a first plunger inserted in the barrel; (iii) a second syringe barrel having a chamber defining a second volume therein; (iv) a second plunger inserted in the second barrel; (v) a manifold attached at a distal end of the device, the manifold having a first leg communicating with the chamber of the first syringe barrel, a second leg communicating with the chamber of the second syringe barrel, and a common conduit interconnecting the first and second legs allowing for passage of components passing through the legs from the respective chambers of the first and second syringe barrels;
a sclerosing agent loaded in the first syringe barrel for use in pleurodesis, a flushing fluid loaded in the second syringe barrel for use in flushing a catheter through which the sclerosing agent is delivered to a patient;
instructions provided for each kit after the patient has been diagnosed with a pleural effusion to be treated, the instructions including directions for a caregiver to administer a selected amount of at least one sclerosing agent over a selected time period.

8. A kit, as claimed in claim 7, wherein:
said instructions further include a description for administering the sclerosing agent in a predetermined sequence in which the plungers of the first and second barrels are selectively activated according to the instructions in which the first and second barrels include markings corresponding to the predetermined sequence.

9. A kit, as claimed in claim 7, wherein:
said instructions further include a description for administering according to the predetermined sequence in which the plungers are activated to deliver predetermined amounts of the sclerosing agent and flushing fluid from said first and second barrels.

10-18. (canceled)

19. An electronic delivery device for delivering selected sclerosing agents for treatment of pleural effusions, comprising:
a housing;
a plurality of chambers placed within the housing, each of the chambers having a selected medicament and/or flushing fluid preloaded therein;
at least one actuator communicating with at least one of the chambers for evacuating contents of a selected chamber;
at least one fluid passageway connected to the plurality of chambers for transferring contents of the selected chamber out from the device; and
a microcontroller communicating with the at least one actuator for evacuating contents within the selected chamber by action of the actuator.

20. A device, as claimed in claim 19, further including:
a start button to initiate a sequence of fluid delivery as controlled by said microcontroller.

21. A device, as claimed in claim number 19, further including:
a check valve associated with at least one of said chambers to prevent backflow.

22. A device, as claimed in claim 19, further including:
a fluid delivery valve communicating with said plurality of chambers to control flow of fluid from the device.

23. A device, as claimed in claim 19, wherein:
said microcontroller is programmed for operation to enable delivery of a selected sequence of medicaments and flushing fluid from the chambers.

24. The syringe device of claim 1, wherein the first contents comprise a sclerosing agent.

25. The syringe device of claim 1, further comprising instructions provided with the syringe device, the instructions including directions for a caregiver to administer a selected amount of at least one sclerosing agent over a selected time period.

26. The syringe device of claim 25, wherein said instructions further include a description for administering according to the predetermined sequence in which the plungers are activated to deliver predetermined amounts of the sclerosing agent and flushing fluid from said first and second barrels.

27. The syringe device of claim 1, further comprising a check valve associated with at least one of said chambers to prevent backflow.

28. The syringe device of claim 24, further comprising a flushing fluid loaded in the second syringe barrel for use in flushing a catheter through which the sclerosing agent is delivered to a patient.

29. The kit of claim 7, further comprising a check valve associated with at least one of said chambers to prevent backflow.

30. The electronic delivery device of claim 19, wherein the device comprises an actuator for each of the plurality of chambers.

31. The electronic delivery device of claim 19, wherein a check valve is provided in association with each of the plurality of chambers.

32. The electronic delivery device of claim 19, wherein the at least one actuator is in communication with a plunger head.
